(19) United States
(12) Patent Application Publication
(10) Pub. No.: US 2023/0115195 A1
(43) Pub. Date: Apr. 13, 2023

(54) PROSTHETIC LINER AND ADDITIVE MANUFACTURING SYSTEM, METHOD, AND CORRESPONDING COMPONENTS FOR MAKING SUCH PROSTHETIC LINER

(71) Applicant: OSSUR ICELAND EHF, Reykjavik (IS)

(72) Inventors: Rowan CAIN, Reykjavik (IS); Stefan Teitur HALLDORSSON, Reykjavik (IS); Ivar OLAFSSON, Reykjavik (IS); Sigurdur ASGEIRSSON, Reykjavik (IS); Andri ORRASON, Reykjavik (IS); Stefan Orn STEFANSSON, Reykjavik (IS); Halldor EINARSSON, Reykjavik (IS); Hafsteinn JONASSON, Reykjavik (IS)

(21) Appl. No.: 17/961,842

(22) Filed: Oct. 7, 2022

Related U.S. Application Data
(60) Provisional application No. 63/253,240, filed on Oct. 7, 2021.

(51) Int. Cl. A61F 2/78; B29C 64/209; B29C 64/245; B29C 64/241; B33Y 80/00; B33Y 30/00

(52) U.S. Cl. CPC A61F 2/7812; B29C 64/209; B29C 64/245; B29C 64/241; B33Y 80/00; B33Y 30/00; B29L 2031/7532

(57) ABSTRACT

An additive manufacturing system and method for making components having filaments formed by elastomeric materials. A liner includes the filaments formed by an elastomeric material and is adapted for a prosthetic device system. The filaments form an elastomeric lattice structure and solid layers or features and define a ventilated structure permitting a transfer of air and moisture from an interior volume of the liner to an exterior or ambient liner. The liner may incorporate an adhesive and a textile layer secured to the elastomeric lattice structure and further define recesses and other features for improving a liner.

### CROSS-REFERENCE TO RELATED APPLICATIONS

This application incorporates by reference U.S. Patent Application Publication No. 2021/0137708, published May 13, 2021, U.S. Patent Application Publication No. 2020/0147875, published May 14, 2020, and U.S. Patent Application Publication No. 2020/0146850, published May 14, 2020.

### FIELD OF THE DISCLOSURE

The disclosure relates to the field of additive manufacturing and, more particularly, to an additive manufacturing system, method, and corresponding components for making structures based on filaments and elastomeric materials.

The disclosure also relates to medical devices, such as prosthetic or orthopedic devices, formed according to the method. For example, an exemplary embodiment is a liner, sleeve, or sock, generally referred to herein as a “liner,” for suspension comfort in a prosthetic device system. The exemplary embodiments are formed from an elastomeric lattice structure and solid layers creating a ventilated structure permitting a transfer of air and moisture from an interior volume of the liner to an exterior or ambient liner.

### BACKGROUND

Additive manufacturing is an increasingly important manufacturing method, comprising numerous uses across industries. Additive manufacturing, also known as “3D printing,” is regarded as a transformative method for industrial production that facilitates the production of a three-dimensional article from a material according to a computer-aided design (CAD) of a definitive article by computer-aided manufacturing (CAM). In this sense, additive manufacturing is a digital revolution of analog manufacturing processes. Efforts have been made to apply additive manufacturing to articles formed from numerous materials, including polymeric materials, a subset of which are elastomeric materials.

Additive manufacturing of elastomeric materials, including silicone, is limited by several factors. In many existing systems, the fluidity of the elastomeric material requires the provision of a vat of liquid elastomeric material or precursor, in which a nozzle deposits curing agents to form a solid article from the liquid elastomeric material in situ, with leftover elastomeric material drained and washed away after the formation process is completed. Other additive manufacturing systems require a low- or room-temperature curing or vulcanizing elastomeric material so that the mass of elastomeric material quickly cures and does not deform during the formation process, as adding multiple layers of elastomeric material may not be accurately performed if the elastomeric material is uncured. Yet other additive manufacturing systems require that individual, discrete beads or droplets of elastomeric material are added one at a time to build a solid three-dimensional elastomeric structure from the base up.

Existing systems for elastomeric additive manufacturing, including those that utilize silicone, compromise the structural quality of the final product by using low viscosity, low-temperature-curing materials to enable the deposition process. In addition, it is not known in the art how to provide a smooth, consistent texture of deposited material having desired material properties. In medical uses, existing additive manufacturing systems preclude the additive manufacturing of medical-grade silicone, having the requisite strength, biocompatibility, and elasticity of conventionally manufactured medical products. Therefore, existing additive manufacturing systems cannot meet the demand for articles made from medical-grade silicone that can exhibit the mechanical and chemical properties obtained from existing articles, including medical devices formed by other conventional manufacturing methods such as molding and extrusion.

Silicone is a desirable elastomeric material in healthcare use due to its biocompatibility and long history of implanted medical devices. In addition, due to confirmatory biological testing, using existing medical-grade silicone materials is desirable to reduce the time from concept to market. Despite its accepted use in healthcare, silicone materials are thick and viscous. These silicone materials require high pressure to be injected into molds to manufacture a precise article, such as through injection molding and transfer molding processes. Challenges are imposed in additive manufacturing because it is difficult to precisely extrude significantly viscous silicone onto a substrate into a definitive shape with high pressure if no mold is employed while accounting for curing and shrinkage rates, as the silicone often deforms, sags, or otherwise loses its desired shape before curing.

While silicone materials can be processed and formed in small batches in a design phase, difficulties arise when scaling up the production of silicone as not only is its viscosity difficult to manage, but other factors must be considered, including curing temperature and time, entrapment of air or bubbles, shrinkage, the mixture of parts, and cross-linking to manufacture medically-accepted articles.

Silicone is a thermoset polymeric material that will cure into its given shape of a strong, dimensionally stable, heat- and chemical-resistant article, but such advantages also require that the silicone's structure cures must be made correctly at the onset as later adaptation is typically not feasible. This limits the customizability of elastomeric structures formed through additive manufacturing. Any additive manufacturing process on a commercially scalable level should preserve the mechanical properties of a cured silicone, such as toughness, elasticity, and other properties desirable in a medical-grade silicone article, while offering high throughput and precision.

Existing systems for additive manufacturing may provide for only a monolithic or single-property structure, as only a single grade or blend of material can be deposited. The structures and functions of additive-manufactured articles are limited to what can be achieved through a single material property. There is a need for an additive manufacturing system that can accurately deposit material having different properties to attain a final product with desired properties in desired regions.

Another problem of existing manufacturing systems is that many are limited to depositing a single discrete bead of elastomeric material at a time, limiting the construction of 3D-printed articles to discontinuous structures that are a sum of individual drops or beads rather than comprising smooth and continuous layers, filaments, or structures with varying properties.

Many production and manufacturing methods are limited to providing a mold in which elastomeric material may be injected and thereafter cured to attain a desired shape and properties. These conditions considerably limit the design and manufacturing flexibility when preparing an article. Because existing methods are limited to processes that deposit discrete beads or inject elastomeric material into negative molds, there is a need for a system that can deposit filaments of elastomeric material to form a structure with desired properties at desired locations.

Existing systems are directed to implementations where an article is built from the bottom up and only in cartesian coordinates. In other systems, the effects of gravity on uncured or partially cured polymer materials limit the dimensions of the article, as too much material added to the article, causes distortions from gravity, particularly combined with the effects of viscosity and curing rates. There is a need for an additive manufacturing system that overcomes the effects of gravity and allows for additive manufacturing of articles in multiple dimensions.

Solutions that attempt to perform additive manufacturing on a rotating build surface or substrate do not provide for manufacturing medical-grade silicones, which require particular viscosities and cure rates, but rather as limited to systems that utilize shavers or cutters that remove extra, unwanted deposited material. These systems also allow material to drip or fall away from the substrate. There is no teaching of using a rotating substrate that achieves desired printing of medical-grade structures from silicone without cutters and dripping configurations to conductive negative manufacturing procedures.

There is a need for an additive manufacturing system that overcomes the limitations of existing systems, namely that low-quality elastomeric materials are used to enable deposition limited to depositing discrete beads, that properties of materials are monolithic and cannot be dynamic to account for different structural and functional needs at different parts or components of an elastomeric additive-manufactured article, and that the methods for additive manufacturing are limited to bottom-up approaches, with gravity effects unmitigated and unaddressed. It is highly desirable to use known silicone materials having confirmatory biological testing in additive manufacturing to create precise silicone-based structures suitable for medical devices.

Liners are widely known and are an interface between a residual limb and a prosthetic socket, allowing a user to comfortably and safely wear the prosthetic socket and prostheses attached thereto, such as prosthetic limbs. Liners may provide cushioning between the end of the residual limb and the prosthetic socket, protecting the limb from developing pressure points as a user's weight is applied to the hard components of the prosthetic socket during use. Liners may also provide improved pressure distribution along the residual limb and the prosthetic socket. In vacuum suspension-type prosthetic systems, a liner may protect the residual limb from exposure to an elevated vacuum for extended periods.

Polymeric, particularly elastomeric, materials are commonly used for constructing liners. For example, a medical-grade silicone may be used that is naturally compatible with human tissue and resistant to fluids and bacteria, reducing the risk of infection. Despite limitations on breathability, these liners often remain fresh and odor-free after each use and have lasting strength and thickness despite repeated use. But many liners may not achieve such desired results upon repeated use, depending on the user's characteristics.

An elastomeric material may be preferred, although not limited, for constructing the liner because it has inherent elasticity that conforms to a residual limb. In addition, the liner's elasticity may be tailored to inhibit elasticity in different directions, such as axially, but enhanced in one direction (radially) relative to another direction such (axially).

Normally a liner is constructed by molding the elastomeric material between male and female molds to form a solid layer of an elastomeric material that may closely encapsulate the residual limb. The elastomeric material may be extruded into a predetermined shape. The liner is created either by molding or extrusion as having a fixed cross-section profile without adapting the molded or extruded part profile.

This fixed cross-section profile is generally a solid mass of elastomeric material that is both vapor- and liquid-impermeable. The solid layer is formed cohesively as a monolithic body. To provide sufficient cushioning and protection of the residual limb, such liners typically comprise a relatively thick layer of fluid-impermeable elastomeric material. The thickness may be increased at a distal end of the liner to provide additional cushioning at the point of the liner where the user's weight is most pronounced against the prosthetic socket.

Because the liner is constructed from a unitary wall or solid layer of elastomeric material, usually formed or cured of a liquid resin poured into the molds or extruded into shape, the material may have uniform properties throughout the body of the liner or simplified properties among various components to the liner (e.g., a taper in thickness). An example of a method for manufacturing a liner is found in U.S. Pat. No. 6,626,952, issued on Sep. 30, 2003, and an example of a liner having multiple components or properties is found in U.S. Pat. No. 6,136,039, issued on Oct. 24, 2000, each of which is incorporated herein by reference.

A common practice is to attach a textile material to an exterior surface of the liner, the textile material having defined properties that may provide customized or desired features at specific locations. The solid elastomeric layer may be cured against the textile material, which requires pre-processing steps, such as sewing and shaping, to have desired properties. One example of the time-consuming and cost-increasing pre-processing steps is stitching a distal seam in a textile tube to shape the textile tube into a liner shape. A liner may provide other components, such as a hard distal end cap.

Stitching and securing a textile to a liner body of an elastomeric material and additional liner components may cause pressure points when worn by a user and pressed against a hard socket. Efforts have been made to minimize such effects, as in U.S. Pat. No. 9,770,891, issued on Sep. 26, 2017, incorporated herein by reference. Still, attention is still desired to simplify processes that provide such textile or other components to a liner body and minimize pressure points.

A known problem in liners is the buildup of moisture and heat between the residual limb and the liner, leading to discomfort, unpleasant odors, “milking,” “pistoning,” and tissue breakdown. For example, medical-grade silicone is hydrophobic because it is vapor- and liquid-impermeable. Sweat may build up between the residual limb and the liner, which may cause slippage of the liner from the residual limb and discomfort, affecting suspension and making skin more prone to breakdown. These drawbacks may lead to a risk of non-compliant use of the prosthetic system or even catastrophic failure of the prosthetic system during use.

Up to 72% of amputees experience a reduced health-related quality of life because of heat and sweating. The impact of sweat and heat on the quality of life for trans-femoral amputees is, therefore, significant. The most common complaints of amputees are perspiration and warmth occurring while wearing a liner. Conventional solid-walled liners impair their occlusive properties, the natural skin regulation mechanisms for humidity and heat management.

There is a balance between providing a liner with sufficient cushioning and thickness to protect the residual limb from harmful extended contact with hard or rigid surfaces and providing a breathable liner to mitigate heat and moisture buildup. A concern arises in whether the liner can maintain the same strength, thickness, compression, and general functionality in a liner having a ventilated structure as in a conventional solid-walled liner. Likewise, there is a desire to maintain the liner constructed from an approved and accepted medical-grade elastomeric material, such as silicone.

Efforts to bridge this gap have included providing wicking layers or absorbent materials within the silicone layer or between the silicone and textile material, which may increase the cost and complexity of constructing a liner. An example of such efforts is the U.S. Pat. No. 9,629,732, granted on Apr. 25, 2017, and incorporated herein by reference. However, efforts to provide apertures, or wicking layers and absorbent materials, may impair a liner's functionality or result in a liner having inferior mechanical properties relative to a conventional solid-walled liner. In addition, such past ventilated liners may prevent or preclude other desirable features in liners, such as external surface peripheral profiles, as in U.S. Pat. No. 7,118,602, issued on Oct. 10, 2006, and seal systems as in U.S. Pat. No. 9,066,821, issued on Jun. 30, 2015, each reference being incorporated herein by reference.

Despite these efforts in the patent literature, there are few commercially available liners with a breathable structure capable of sweat management. Other sweat-preventing interventions are tap water iontophoresis, talcum, wiping residual limb, and airing out a residual limb. The injection of Botulinum toxin has also been reported to be effective but comes with a greater clinical intervention and provides relief only temporarily.

A liner still needs to achieve the structural and cushioning benefits of solid-walled, conventional liners but can mitigate the buildup of heat and moisture while preserving its construction from a medical-grade material and accommodating various features common in conventional liners.

Another problem in existing systems and methods for producing liners is the difficulty and cost of providing a custom-fitted prosthetic system with features that correspond to the needs of different portions of the residual limb. Each residual limb has unique dimensions and shape, and the efforts of a trained prosthetist must assess a user's needs should the user's needs be outside normal shapes and sizes of liners. Individuals may have different bony mass structures and soft tissue, depending on how the residual limb occurred. It is difficult to meet the individual residual limb's unique limb shape and needs, particularly as, due to swelling or weight change, the dimensions and needs of a particular user may be dynamic and change.

As it is difficult to achieve the structural and functional needs of each residual limb, it is desirable to provide a liner that can meet the demands of each user, whether the liner is for lower or upper extremities and whether the user requires an elevated vacuum, seal-in expulsion, and locking suspension systems. Custom liners may be provided for amputees of all lifestyles and activity levels, and there is difficulty meeting the demands of all such individuals with standard conventional-sized liners. Individuals may require material additives for easier donning and doffing, skin-treatment additives, and desired conventional liner features in a custom-fitted liner.

Because many medical devices having elastomeric materials such as medical-grade silicone are formed by injection molding, where a silicone resin is injected into a space defined by a negative mold of the medical device, most medical devices do not have a desired degree of customized properties based on the functionality of different regions of a user's body but have uniform properties throughout. In the example of a liner, however, it may be desired to have more elasticity at and behind the knee compared to above, below, and to the sides of the need, or a different degree of breathability may be desired at regions proximate active muscle groups that generate more heat and fluid. Thus, there is a need for a medical device that provides custom properties at desired locations around the medical device rather than uniform properties.

There is a need for a liner tailored to an individual user's demands while offering accommodation for conventional liner features. More generally, medical devices are needed to be constructed from elastomeric materials that offer a desirable balance of breathability and mechanical properties to withstand the device's normal daily use. Despite prior efforts and alternative treatments, such desired liner should reduce the moisture on skin over commercially available liners, increasing perceiving improvements in stability and suspension, offering equivalent comfort over known liners, and offering overall improved skin health.

### SUMMARY

The additive manufacturing system, method, and corresponding components for making elastomeric structures of the disclosure advantageously provide a system for providing material in desired quantities and at desired locations of an article with an improved dispensing and deposition apparatus, resulting in smooth, continuous depositions of beads, filaments, or layers of material with a controlled variation of desired properties and at desired locations.

Building on the existing additive manufacturing system, method, and corresponding components for making elastomeric structures of the disclosure, as taught in U.S. Patent Application Publication No. 2020/0147875, methods are provided herein for correcting for the possibility of an offset of a mandrel upon which material is deposed. According to the disclosure, a mandrel serves as a deposition substrate and is arranged to rotate. The deposition apparatus deposits material in different patterns and orders along a surface or built upon a surface of the mandrel.

As the mandrel may become off-center or wobble after successive use, a method is provided for calibrating the deposition apparatus to accommodate variances in the mandrel's rotation. Before each formation of an article, such as a liner, a mandrel offset correction may be provided by controlled corresponding movement of at least one nozzle of the deposition apparatus. The offset correction enables more consistent deposition of the elastomeric material, thereby creating a more uniform structure and assuring adherence of various layers of the article.

According to an aspect of the method, different nozzles may be employed at different stages or for different layers or segments of the article. For example, a single nozzle may form a skin contact layer in a prosthetic liner. According to this layer, there may be solid sections, such as sections devoid of apertures or other ventilation features, and may form different structures to the liner. For example, the nozzle may be positioned above the mandrel or within a predetermined range for dispensing material at a higher speed to cause loops or otherwise formed filaments or structures to be deposited on the mandrel.

According to another aspect of the method, the deposition apparatus may include at least two nozzles for simultaneous material deposition on the mandrel. Such nozzles are controlled and calibrated together to deposit material simultaneously, reducing the time to complete the layer. It should be kept in mind that by layer, such layer may include multiple sublayers of filaments or structures, as described in U.S. Patent Application Publication No. 2020/0146850.

According to another aspect of the method, the article, as in a prosthetic liner, may be formed by a deposition of materials forming layers having different heights and/or features. For example, the prosthetic liner may be formed by the deposition of an elastomer forming recesses for accommodating features of the prosthetic liner that are secured to the liner post-formation according to the methods of additive manufacturing.

The balance of strength, comfort, breathability, and other desired properties of elastomeric and other polymer-based and especially elastomer-based medical-grade materials in medical devices, such as prosthetic and orthopedic devices, is addressed in embodiments of the disclosure. These embodiments exemplify a liner comprising discretely and continuously deposited layers of the medical-grade elastomeric material, such as silicone, used in conventional liners while maintaining at least equivalent mechanical strength and other mechanical properties of such conventional liners.

While such liners may be constructed from the same medical-grade elastomeric material and possess the same mechanical and chemical properties of conventional liners, the structure of the embodiments of the disclosure provide improved cushioning, moisture removal, and/or breathability over known conventional liners. The embodiments may be provided combined with textile covers, reinforcement layers, material additives, and other desired features in conventional liners while having the aforementioned improved features. While medical-grade elastomeric material is discussed, it will be understood that the disclosure is by no means limited to medical-grade material and may make use of any suitable material.

The exemplary embodiments offer a reduction of moisture during increased perspiration, leading to perceived improvements in stability and suspension, improved or at least equivalent comfort over conventional liners, and improved skin health.

The exemplary embodiments possess characteristics that can be extended to a wide range of medical devices, including prosthetic or orthopedic parts, medical implants, medical tubing, prostheses, and other parts or devices. The characteristics may be adapted according to desired properties or needs and customized to address users' needs. For example, the characteristics of the embodiments can be used in devices made by known medical-grade elastomeric materials, removing the necessity for material approval and streamlining regulatory acceptance.

Exemplary liner embodiments are arranged to effectively manage perspiration formed by a limb, prevent slippage of the liner on the limb, and provide suitable cushioning for a limb. The exemplary embodiments described are discussed and shown within the context of a liner in a prosthetic system for use with a hard socket. However, the disclosure is not limited to such a prosthetic embodiment or the exact uses described and embraces any use requiring perspiration management, prevention of slippage, cushioning of the limb, or any other structural and/or functional benefit that may derive in whole or in part from the principles of the disclosure. Principles described herein may be extended to any prosthetic, orthopedic, or medical device and are in no manner merely limited to liners.

In an exemplary embodiment, a liner advantageously bridges the gap between a solid-layer wall liner's strength and the need for breathability while using a medical-grade material. The liner may be customized to have features at particular locations corresponding to individual users' needs, minimizing cost and complexity of manufacturing, and offering physical structure and functionality that benefit different requirements. The liner is just an example of the different structures that can be manufactured and configured according to principles described herein.

In an example, a textile sleeve having an end cap may be secured over a distal end of an elastomeric body formed according to the methods, in which the end cap is configured and dimensioned to fit within a recess formed at a distal end of the prosthetic liner. The textile sleeve is secured to the prosthetic liner elastomeric body. The elastomeric body may be pretreated with a glue layer, likewise deposited by the deposition apparatus such that a layer of an adhesive adapted to secure a textile sleeve to the elastomeric body.

The end cap may fit within the recess so as not to protrude beyond a sidewall formed by the prosthetic liner. The end cap may be formed from a more rigid material thereby adding improved stability to the distal end of the prosthetic liner. Additional features carried by the textile sleeve, such as a seal, or other components in addition to the textile sleeve may fit within corresponding recesses formed by the prosthetic liner formed by additive manufacturing.

In a variation, a textile layer may be secured to a discrete section of the elastomeric body, and such discrete section may include a recess adapted to receive the textile layer so that the textile layer is flush with an outer surface of the elastomeric body outside of the discrete section.

The end cap may be adapted with an outlet valve formed at a distal end of the prosthetic liner. The outlet valve may be formed, at least in part, by a hole extending through the distal end cap to an interior cavity of the prosthetic liner. The outlet valve is preferably a one way valve.

These and other present disclosure features will become better understood regarding the following description, appended claims, and accompanying drawings.

### BRIEF DESCRIPTION OF THE DRAWINGS

FIG. 1 is a schematic elevational view of an embodiment of a liner.

FIG. 2A is an exemplary view of a step for calibrating a deposition apparatus relative to a mandrel.

FIG. 2B is another exemplary view of a step for calibrating a deposition apparatus relative to a mandrel.

FIG. 2C is an exemplary view of a deposition apparatus relative to a mandrel.

FIG. 3 is an exemplary view of depositing a base layer in a prosthetic liner.

FIG. 4 is an exemplary view of operating a deposition apparatus having at least one nozzle.

FIG. 5 is an exemplary view of depositing an adhesive layer to an elastomeric body of a prosthetic liner on a mandrel.

FIG. 6 is a perspective view of a distal end of an elastomeric body having at least one recess formed during a deposition process.

FIG. 7 is an exemplary view of applying a textile layer to the elastomeric body of FIG. 6.

FIG. 8 is a perspective view of a distal end of a prosthetic liner having an end cap.

FIG. 9 is a perspective view of a distal end of a prosthetic liner having one-way valve.

FIG. 10 is an exploded perspective view of the prosthetic liner of FIG. 9.

FIG. 11 is an elevational view of another embodiment of a prosthetic liner having a textile band in an inverted configuration.

FIG. 12 is an elevational view of another embodiment of a prosthetic liner having solid thickness sections in an inverted configuration.

The drawing figures are not necessarily drawn to scale. Instead, they are drawn to provide a better understanding of the components and are not limited in scope but to provide exemplary illustrations. The figures illustrate exemplary configurations of a liner and in no way limit the structures or configurations of a liner and components according to the present disclosure.

### DETAILED DESCRIPTION OF VARIOUS EMBODIMENTS

Embodiments of a liner overcome the limitations of existing liners by providing a liner structure that advantageously allows for breathability, minimizing the buildup of heat and moisture, without sacrificing the robustness, cushioning, strength, and other advantageous features of solid-walled liners. The liner provides for discrete zones of different features that better address the needs of individual users and the shapes and needs of different residual limbs.

According to the methods and systems of the disclosure, partially cured or uncured medical-grade elastomeric material, such as silicone, is sequentially deposited onto a substrate by a nozzle or similar device from a material source in a controlled manner according to computer control to define a definitive shape, such as an elongate or continuous filament. The deposited elastomeric material may be a thermoset material such as silicone or thermoset polyurethane, or any other suitable polymeric material, resulting in curing after it has been deposited from a nozzle. The additive manufacturing system of the disclosure can deposit elastomeric material with a preferred blend of elastomeric materials to attain the desired property at the desired location along or within a medical device so that a continuous filament may have different properties, compositions, and shapes at different locations along its length.

Examples of medical-grade silicone are obtainable from NuSil Technology of Carpinteria, Calif., under product designations CF13-2188, MED-4901, MED-6340, or MED-6345. Other silicone compositions can be used, and the embodiments herein are not limited to the exemplary silicone materials but rather may be formed from other suitable polymeric or elastomeric compositions such as polyurethane, block copolymer, etc.

Different structures of a cushion layer or the layers described may be formed according to the disclosure in co-pending U.S. application Ser. No. 16/680,959, particularly those of lattice structure or solid structures formed by filaments from an elastomeric material. Any layer of the following liner described can be made or have a structure according to the co-pending applications associated with a lattice or solid structure defined by a plurality of discretely formed filaments.

Exemplary liner embodiments are arranged to effectively manage perspiration formed by a limb, prevent slippage of the liner on the limb, and provide suitable cushioning for a limb. The exemplary embodiments described are discussed and shown within the context of a liner in a prosthetic system for use with a hard socket. However, the disclosure is not limited to such a prosthetic embodiment or the exact uses described and embraces any use requiring perspiration management, prevention of slippage, cushioning of the limb, or any other structural and/or functional benefit that may derive in whole or in part from the principles of the disclosure. Principles described herein may be extended to any prosthetic, orthopedic, or medical device and are in no manner merely limited to liners.

In an exemplary embodiment, a liner advantageously bridges the gap between a solid-layer wall liner's strength and the need for breathability while using a medical-grade material. The liner may be customized to have features at particular locations corresponding to individual users' needs, minimizing cost and complexity of manufacturing, and offering physical structure and functionality that benefit different requirements. The liner is just an example of the different structures that can be manufactured and configured according to principles described herein.

According to an exemplary embodiment, the liner has a first or proximal end, a second or distal end, and a tubular liner body defined between the first and second ends. The liner body preferably comprises a facing or base layer formed from an elastomeric material, such as silicone, and has an inner surface extending along with an interior of the tubular liner and defining a periphery thereof. The facing layer defines a plurality of openings extending, preferably through a thickness thereof. As the facing layer is intended to secure against a user's skin about the residual limb, the facing layer may have a more combined solid surface area than a combined area of the plurality of openings to provide an effective skin interface. The facing layer's inner surface is preferably smooth because it has a generally uniform surface elevation aside from the openings.

The facing layer may comprise a plurality of filaments integrally adjacent to and/or chemically bonded, preferably without adhesive, to one another to form a continuous solid layer. The filaments are aligned with one another and are chemically bonded along their length to an adjacent filament without a gap. Such a structure can be formed to constitute a film that is both vapor and liquid impermeable. One filament may be continuously formed against an adjacent filament, whereas the adjacent filament may be formed with gaps along its length, with yet another filament on an opposing side of the adjacent filament to form an apertured or ventilated layer; however, such apertured or ventilated layer has apertures positively formed without mechanically or chemically perforating a solid layer to form such apertures. In embodiments, a solid or continuous film or layer may be formed, and then the material may be removed in any suitable manner to define the apertures.

A first layer formed from an elastomeric material is secured to an outer surface of the facing layer (so the facing layer is secured to the inner side of the first layer) and comprises a first set of interstices or interstices having axes corresponding to axes of the openings of the facing layer. The first layer comprises a first sub-layer, including a plurality of first filaments arranged in a first direction and a second sub-layer, including a plurality of second filaments arranged in a second direction. The second sub-layer overlaps the first sub-layer and forms the plurality of interstices therebetween. An elastomeric material's material properties forming the facing layer may differ from the material properties of an elastomeric material forming the first layer. The facing layer may include a skin care additive such as a moisturizer, an antimicrobial composition, aloe vera, or otherwise, whereas the first layer may not, and vice versa.

Each filament may have a uniform cross-section extending along its length in a predetermined shape in a preferred embodiment. Each filament is formed discretely and extends continuously relative to adjacent filaments. These discretely formed filaments may constitute basic building blocks of the liner or medical device structure. While the preferred embodiments display the filaments as arranged in a lattice-like network, they may be arranged relative to one another at varying distances and orientations relative to one another. The lattice-like network defines a plurality of interstices between the filaments, leading to passages for transferring air and moisture through the lattice-like network. The filaments may be arranged relative to one another in an infinite number of coordinates relative to one another in x-, y-, z- planes and/or coordinates. A cross-section of the filaments may be modified to resemble any desired geometric shape such as a square, rectangle, triangle, or circle, while an exemplary shape is a generally round configuration. The cross-section may be asymmetric and be different at different lengths or locations of a continuous filament.

The first and second sub-layers of the first layer are preferably chemically and integrally bonded to one another and might be formed from the same elastomeric material but are compatible materials nonetheless to assure bonding. Likewise, the facing layer and the first sub-layer are chemically bonded to one another from compatible materials. In this manner, the sub-layers integrally form an inseparable and continuous structure bonded together to act mechanically as a monolithic structure. By chemically and integral bonding, a preferred embodiment is without an adhesive, so the filaments are bonded together as the elastomeric material defining the filaments is a curing material and sufficiently fluid for the layers to at least slightly blend into one another at an interface thereof; however, it is not outside the scope of the disclosure to use an adhesive, a primer, or any other suitable means.

Additional layers may be secured to a second or outer side of the first layer (i.e., a second layer formed similarly to the first layer and secured to the first layer). These additional layers are preferably formed together as an inseparable and continuous structure to act mechanically as a monolithic structure. The second layer may be chemically bonded to the second sub-layer of the first layer and comprise a plurality of interstices with axes corresponding to the first layer's interstices.

A textile or fabric layer may be secured to the outer periphery of the first layer or the additional layers. It may be breathable to permit air and moisture passage from the inner surface of the facing layer or interior volume of the liner through an entire thickness of the first layer and additional layers. Hence, an axis extends through each interstice of the first layer, the corresponding interstice of an additional layer, and a respective or corresponding opening of the facing layer. The breathability is not limited to merely passing through a wall thickness, but air and moisture may transfer in all directions within the lattice network of interstices, which define a lattice structure. For example, air and moisture may be channeled to transport through the interstices and out from a proximal end of the liner which may be open to the ambient.

The openings of the facing layer and the interstices of the first layer and additional layers are arranged in a predetermined shape and pattern in a controlled manner. While materials of the base, first, and additional layers may be elastomeric, they may be formed of the same material or different materials. The base, first, and additional layers may have different or similar mechanical properties. The layers may be tailored to different mechanical properties according to the location of the layer relative to the liner. For example, the facing layer may have a lower durometer as a whole than the first layer.

In embodiments, a region corresponding to a joint such as a knee may be formed from materials imparting greater elasticity or breathability than an adjacent region. For example, the facing layer may have an unapertured region comprising a substantial surface area of the facing layer beyond just spacing between apertures, as will be discussed. The unapertured region may comprise a solid patch region corresponding to anatomy of a user, such as a groin area, to avoid possible chafing and skin irritation at sensitive areas of the user.

The materials are preferably compatible materials to allow for chemical bonding, so they are joined permanently to each other and may share at least a blended region in which materials of the layers intermix or interlock to form the permanent chemical bond. Other features, such as seals, volume control pads, cushioning pads, distal caps, etc. may be formed from compatible materials and chemically bonded to or within a thickness of the liner body.

By arranging discretely deposited filaments and layers of materials having different properties, the liner advantageously provides enhanced precision in attaining desired mechanical properties, structures, and functions over existing liners. Inner layers may provide greater comfort through having a lower durometer, for example, while outer layers may have a greater thickness and greater elasticity to provide mechanical strength and desired functional properties. In some embodiments, the discretely deposited layers of material may comprise multilayer depositions, points, or filaments of different materials having different properties.

According to a variation, the filaments may be arranged with co-extruded materials, so two materials are co-axial, with an outer layer formed from a material having a different hardness (or other property) than a material forming the inner layer. Among some reasons, the outer layer can protect a soft inner layer and form strong chemical bonds with adjacent filaments. In embodiments, the elastomer may be co-extruded with textiles such as yarn. In other embodiments, the elastomer may be extruded as a continuous filament with different properties at different locations provided by in-line dosing of additives, for example, the addition of oil at certain locations to achieve a lower durometer. The stretchability of the inner layer can be controlled by the outer layer while permitting the compressibility of the soft inner layer. This embodiment allows the discretely formed filaments to have the advantage of providing multiple types of materials simultaneously. For example, the liner can have properties and advantages of a hard, durable material and the properties and advantages of a soft cushioning material.

The combination or bonding of adjacent filaments can be extended to solid wall portions of the liner that are vapor- and liquid-impermeable solid-walled liners or other medical devices having solid wall portions, or which are solid entirely. Preferably, the solid wall portions may be formed from a plurality of adjacent and abutting filaments, which are also discrete and continuous. The resultant structure is preferably smooth and continuous in a sense there is no identification to the naked eye of each filament of the plurality of discrete filaments, whether mechanical, tactile, or functional. The resultant structure of the adjacent filaments is other filaments having blended chemical bonding by adjacent and abutting filaments in x-, y-, z- planes, and/or coordinates.

In an embodiment, a textile is provided over an outer surface of an elastomeric liner body, and the elastomeric material is used to seal and secure the textile on the liner body. The textile may be placed over the liner body and mechanically interlock therewithin that the elastomeric material of the liner body impregnates the textile, and a discrete portion of elastomeric material is used to close the textile material about the liner body, removing any stitching. This feature is advantageous because the embodiment can avoid uncomfortable pressure points by eliminating seams and stitching. This feature is also advantageous because the textile can be attached to the liner body over many points on the textile, ensuring a strong, durable bond. The manufacturing process is also simplified by the removal of the separate stitching procedure. The textile may be closed and any seam may be reinforced with heat bonded tape.

Because of the controllability of forming the liner according to the structure described above, versatility is provided in forming custom-fitted liners with various features, which are integrally formed or secured to one another. In addition, the liners may be custom formed by a lay-up of compatible materials having different yet compatible properties to accommodate uniquely shaped residual limbs.

For understanding the subsequent improvements and embodiments, a prosthetic or orthopedic device includes a lattice structure defined by a first layer of first filaments discretely formed from a first elastomeric material and overlapping a second layer of second filaments discretely formed from a second elastomeric material. The first and second filaments of the first and second layers, respectively, overlapping and securing to one another at discrete intersections to form a first set of interstices located therebetween in a predetermined pattern. The first and second layers are blended at least in part with one another in a blended region at an interface of the first and second layers. The blended region forms a permanent chemical bond in which the first and second elastomeric materials of the first and second layers of the first and second filaments intermix.

The device forms a proximal end and a distal end, and a body defined between the proximal end and distal end. The body may have a tubular shape defined by a mandrel upon which the filaments are deposited in the form of uncured liquid elastomeric material, such as silicone. The first and second filaments continuously spiral about the tubular shape of the body and the first and second layers are relatively concentric to one another. The first elastomeric material may be different from the second elastomeric material, or formed from the same elastomeric material.

The base layer may be formed from a plurality of first base-layer filaments formed from a third elastomeric material and directly adjacent to one another without interruption and defining a continuous sheet. The plurality of first base-layer filaments blend into one another to form a continuous and contiguous border. The base layer is permanently secured to an inner side of the first layer of filaments by being chemically bonded to the first layer of filaments. The base layer forms a substantially solid film of the third elastomeric material and a solid surface area.

The base layer defines a plurality of apertures formed from shortened segments of second base-layer filaments formed from the third elastomeric material and directly adjacent without interruption to the first base-layer filaments. The first and second base-layer filaments are permanently and chemically bonded to one another. Individual apertures of the plurality of apertures of the base layer may be sized substantially smaller than the interstices of the lattice structure, and are in correspondence with the interstices of the lattice structure. The base layer may be concentric to the first layer. The third elastomeric material may be different from the first elastomeric material forming the first layer. the third elastomeric material includes silicone oil.

As the disclosure is not limited to liners, other medical devices may be formed by medical-grade elastomeric materials, such as silicone, according to the principles described herein from discretely and continuously deposited elastomeric material. These medical devices may be prosthetic or orthopedic parts, medical implants, medical tubing, prostheses, or other devices that employ such medical-grade elastomeric materials.

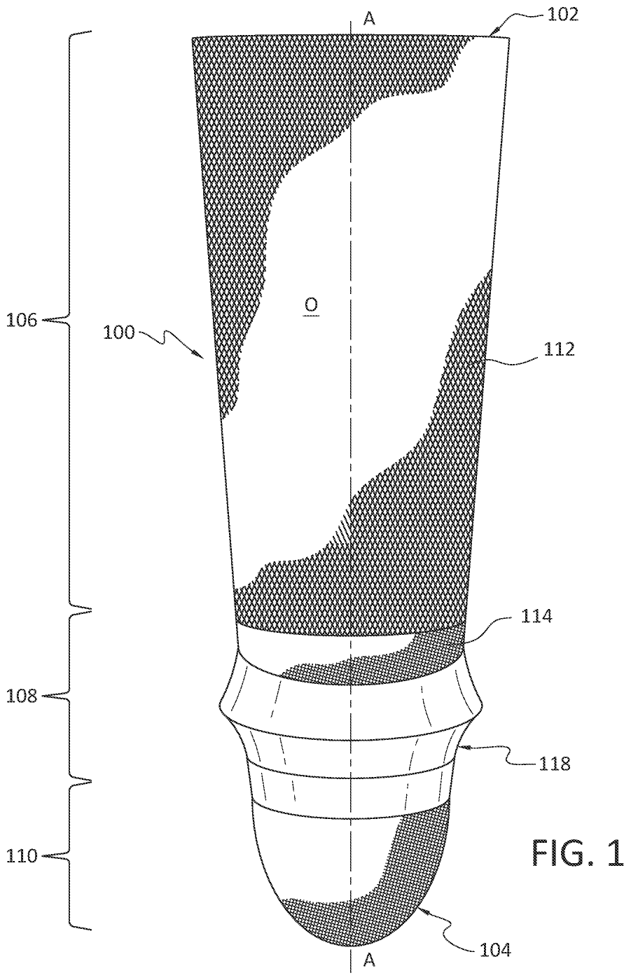

Referring to FIG. 1, an exemplary liner 100 for prosthetic use defines a proximal end 102 and a distal end 104. The liner 100 has a body region 106 extending from the open proximal end 102 distally toward a closed distal region 110 at the distal end 104 along an axis A-A. The liner includes a cushion layer 112 located at least within the body region 106 and is formed from a lattice structure. The lattice structure defines a plurality of interstices or voids between structural filaments or components forming the lattice structure, and such interstices, as inherent in a lattice structure, enable a transfer of air and moisture through the lattice structure. In this embodiment, the cushion layer 112 defines having an outer surface O to the liner but may be arranged with an outer textile cover, as discussed below.

The liner 100 includes a textile layer 114 with a first surface located along a second surface opposite the first surface of the cushion layer 112. A facing layer (not shown) is located along a second surface opposite the first surface of the textile layer 114. The textile layer 114 may be porous, so it is vapor and liquid permeable.

The liner includes a seal region 108 located between the body region 106 and the distal region 110. The seal region 108 has a seal 118 extending radially from the axis A-A relative to the body region 106. The seal may be formed and arranged as of the seals disclosed in U.S. Pat. No. 9,066,821.

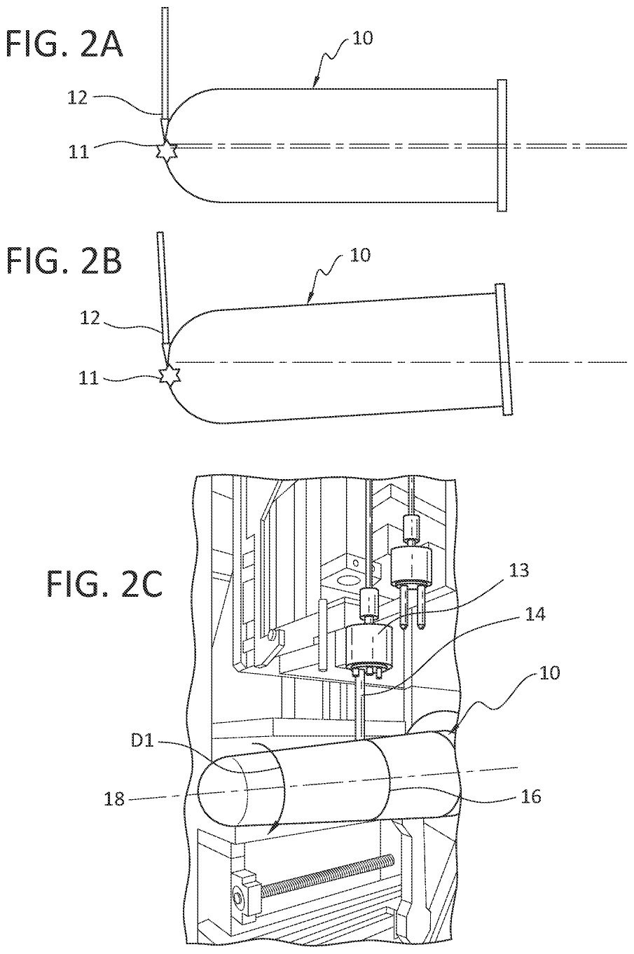

Referring to FIGS. 2A-2C, a mandrel 10 may be used as a substrate upon the elastomeric material is deposited. A mandrel offset correction routine is used for controlling the movement of at least one nozzle of a dispensing apparatus, as shown in U.S. Patent Application Publication No. 2020/0147875. A probe 12 is used for detecting a possible axis offset of the mandrel.

The work offset of all G-Code paths is at a center-tip 11 of a distal end of the mandrel 10. All G-Codes assume that X, Y, Z, and U axis are at a machine zero at this position. The work offset is not the same as the machine zero. Depending on which dispenser and nozzle are used for depositing a material, different work offsets may need to be determined to align the tip of the nozzle of the dispenser apparatus to the correct position.

Two probes may be mounted on the apparatus for rotating the mandrel and supporting the deposition apparatus; a probe on the Y-Axis, which is the axis that moves the mandrel back and forth, and another probe on the Z-Axis, which is the axis that moves the dispenser apparatus up and down relative to the mandrel. All dispenser nozzles are to be probed to the Y-Axis probe. In this manner, the machine knows the coordinates/distances between each nozzle and the Y-Axis probe.

The Z-Axis probe is used to probe the machines mandrel adapter plate and the Y-Axis probe. This arrangement allows the machine to know the coordinates/distances between the Y-Axis probe and the mandrel adapter plate. By knowing the mandrel length, the machine can calculate the work offset for any dispenser nozzle based on the results from the already described probing.

The Z-Axis probe is used to probe the mandrel for measuring the offset and wabble. The small offset and wabble values are added to the movements of Z and U axis while deposition to ensure correct distances of the nozzle to the mandrel at all time during printing. The Z-Axis probe is used to probe the mandrel adapter plates side when no mandrel is mounted, for measuring the slope of the Y-Axis compared to the Z and U-Axis. The small slopes are added to the movements of Z and U axis while printing to ensure correct distances of the nozzle to the mandrel at all time during printing.

FIG. 2C, consistent with the method described in U.S. Patent Application Publication No. 2020/0147875, illustrates a deposition apparatus 13 having a nozzle 14 arranged to deposit layers 16, according to U.S. Patent Application Publication No. 2020/0146850, on the mandrel 10. The mandrel 10 has an offset relative to an axis 18. The dispenser apparatus 13 is arranged to accommodate the offset as the mandrel rotates in direction D1, creating a consistent pattern about the mandrel and assuring proper adhesion of deposited filaments and/or layers on the mandrel.

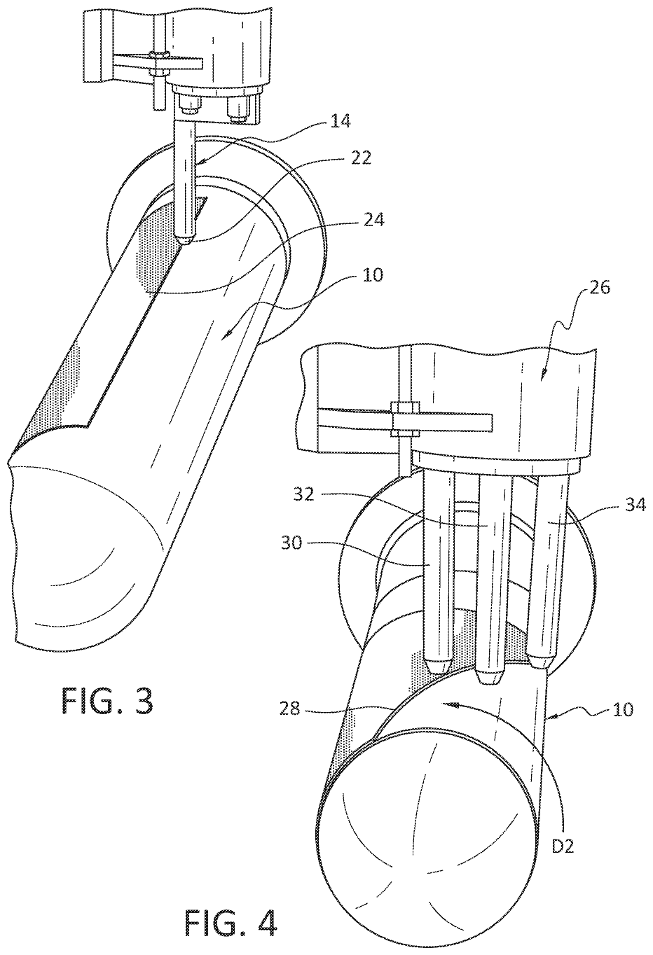

FIG. 3 illustrates an initial deposition of a facing layer 24 on a mandrel 10. The facing layer 24 is the first layer of material deposited on the mandrel 10 by a deposition apparatus 14 having a nozzle 22. The configuration by which the facing layer 24 is deposited on the mandrel 10 may be controlled according to the aforementioned calibration/correction method. Alternatively, the configuration of the facing layer may not be controlled by a calibration method. Rather the facing layer may be computer designed/controlled via G-codes, and the calibration may ensure consistent results when running the G-code.

As is best understood, a G-code is a computer numerical control (CNC) programming language, and is used in computer-aided manufacturing to control automated machine tools, and has many variants. G-code instructions may be provided to a machine controller (industrial computer) that tells the motors where to move, how fast to move, and what path to follow. The two most common situations are that, within a machine tool such as a lathe or mill, a cutting tool is moved according to these instructions through a toolpath cutting away material to leave only the finished workpiece and/or, an unfinished workpiece is precisely positioned in any of up to nine axes around the three dimensions relative to a toolpath and, either or both can move relative to each other. The same concept can be extended to additive methods, as in the methods described herein.

FIG. 4 exemplifies how a deposition apparatus 26 may be equipped with at least two nozzles. In this example, three nozzles 30, 32, 34 for depositing material on the mandrel 10 rotating in a direction D2 for forming a layer 28. The nozzles may be arranged in a predetermined manner to achieve the desired pattern of deposited material and speed up the fabrication process. Of course, other configurations are possible and may be mounted accordingly to the deposition apparatus. The nozzles may move and deposit uncured liquid silicone simultaneously onto the mandrel along the Y-axis to create a helical pattern shown in FIG. 4.

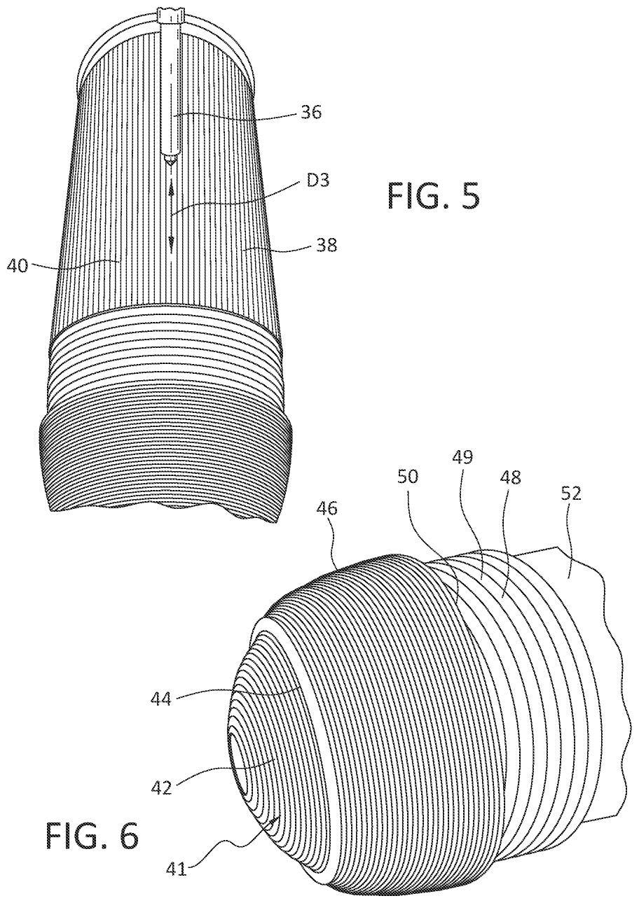

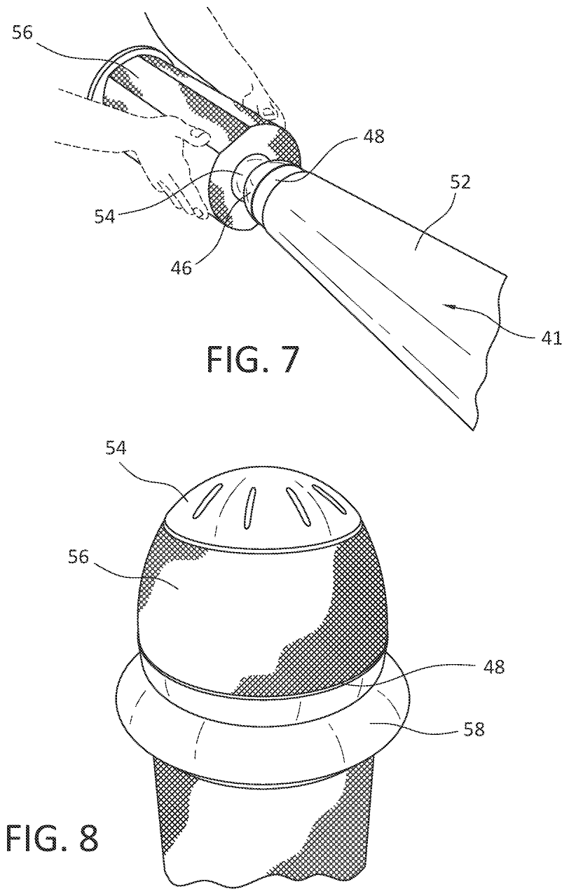

FIG. 5 shows how before applying a textile layer (shown in FIG. 7) to the elastomeric body 38 formed from additive manufacturing, an adhesive layer 40 may be strategically deposited to the elastomeric body 38 from a nozzle 36 extending from the deposition apparatus (not shown). The adhesive layer 40 can be tailored in a pattern, such as longitudinal lines along direction D3, and adapted as needed to ensure the textile layer's sufficient and durable adherence to the elastomeric body.

An adhesive layer may be applied or layered onto at least an outer surface of an outermost surface (defined as being the surface of the elastomeric or lattice body farthest away from the axis of the body), but in a more randomized and/or discontinuous pattern. By discontinuous pattern, it means that the pattern is not formed from a consistently continuous form, such as a thin, continuous and generally non-apertured film or coating, thereby forming an air and moisture impermeable structure, but rather has openings or segments or other forms of material that do not define a solid and continuously solid structure devoid of spacing or openings.

For example, the pattern may be adapted by tighter or wider spacings between the longitudinal lines, or segmented lines adapted to apply over extending non-apertured sections along the outer surface of the elastomeric body, or arranged in a manner so as not to occlude a lattice framework forming at least part of a thickness of the elastomeric body, as taught in U.S. Patent Application Publication No. 2020/0146850. Such deposited adhesive layer with a deposition apparatus is in contradistinction to conventional means for applying an adhesive to an elastomeric body in liners lacking a breathable thickness whereby the adhesive layer is randomly or totally applied to an outer surface of an elastomeric body.

FIG. 6 depicts how the elastomeric body 41 may be formed with recesses about an outer periphery thereof for receiving additional components of the liner not formed by additive manufacturing. In the depicted example, the distal end of the elastomeric body 41 forms a recess 42 at its distal end and is adapted for receiving an end cap, shown in FIGS. 7 and 8. The recess 42 has a depth 44 from an adjacent area 46 outside of the recess 42 and is located more proximal than the distal end, and preferably circumferentially extends about the distal end of the elastomeric body or liner. The depth 44 may be selected according to a thickness of the end cap in that the end cap will fit flush with the distal end of the elastomeric body and not protrude beyond the adjacent area 46.

Another recess 48 may be formed having a depth 50 the same or different from the depth 44, yet configured and dimensioned to receive another component. The recess 48 is axially displaced from the recess 42 in that it is preferably circumferential and located axially closer to the proximal end of the elastomeric body or liner. An exemplary component is a seal element, as shown in FIG. 8. The recess 48 in contrast to the first adjacent area 46 and a second adjacent area 52 on an opposite side of the recess 48.

Either of the recesses 42, 48 may define a surface relief 49 to accommodate the end cap better and/or seal element or any other component. The surface relief may vary according to the component, adherence of the component, or function. The possibility of creating the recesses 42, 48 through additive manufacturing offers a spectrum of options; each recess can be uniquely tailored according to prescribed needs corresponding to the component or other function of the prosthetic liner.

FIG. 7 illustrates a textile layer 56 having an end cap 54 being applied to the distal end of the elastomeric body 41 in FIG. 6. Once the adhesive layer is applied to the elastomeric body 41, the textile layer 56 may be rolled onto the elastomeric body 41. Alternatively or in addition to the adhesive layer, the textile layer 56 may be provided with an adhesive layer to facilitate bonding of the textile layer to the elastomeric body 41. The end cap may be formed from an elastomeric material is an integrally secured to the textile layer either before or after adherence to the elastomeric body.

FIG. 8 shows the distal end of the liner in that the end cap 54 is secured to the elastomeric body and does not protrude beyond the contours of the elastomeric body outside of the recess at the distal end. Likewise, a seal element 58 is shown as corresponding to the recess 48 of the elastomeric body and fits therewithin.

The end cap is formed and defined before engagement with the elastomeric body and is preferably constructed from a stiffer elastomeric material than the material forming the elastomeric body. The end cap is preferably assembled with the textile layer before bonding to the elastomeric body. The end cap to steer or centralize the textile layer when it is stretched over the liner for bonding.

The end cap may inhibit distal elongation of the liner, as it is formed from a substantially more rigid material or is formed more structurally rigid than the elastomeric body distal of the end cap. The textile layer may also have anisotropic properties to inhibit the stretching of the liner. Due to the advantageous method for forming the elastomeric body, the recess enables to tight connection with the end cap, thereby enabling a secure mating of the end cap at a precise location at the distal end of the elastomeric body.

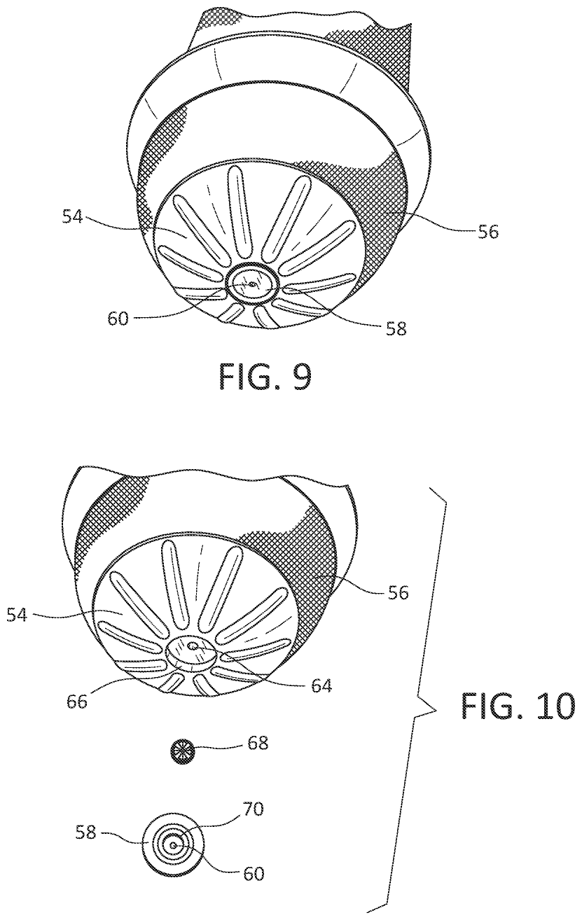

FIGS. 9 and 10 illustrate a valve located at the distal end of the liner. The end cap 54 defines a recess 66 in which a one-way valve 58 is disposed of. The one-way valve defines an aperture 60 communicating with another aperture 64 defined in the elastomeric body. A valve insert 68 is provided in a concavity 70 of the one-way valve 58, permitting air to only be expelled from an interior cavity of the prosthetic liner to the exterior of the liner. Therefore, the air and/or moisture is always expelled from the interior cavity and the residual limb, and a passage of air from the exterior to the interior cavity is prevented. Exemplary valves may include a duckbill or a cross-slit valve.

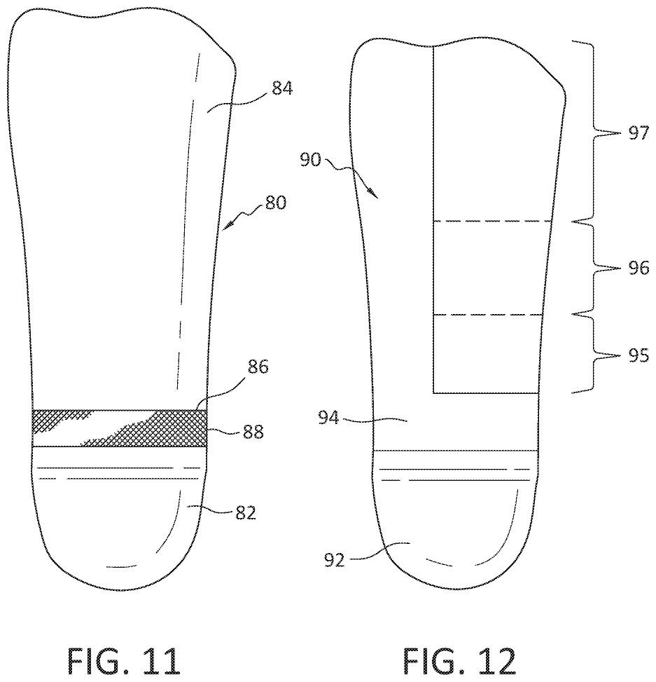

FIG. 11 exemplifies a prosthetic liner 80, including a textile band 88 provided in a circumferential recess 86 formed by the elastomeric body 84. The textile band 88 may be located above a distal end 82 of the liner 80. The textile band may be a wicking material or an absorbent material. As an absorbent material, it may absorb sweat before it is pushed toward a proximal end of the liner.

The textile band can be substituted by other materials providing a designated purpose. For example, the textile band may be formed from an absorbent foam. In another example, the band may be formed from a substantially frictional material (for firm engagement with skin of a residual limb) to effectively form a seal or resist pistoning of the liner in the socket. The band may be segmented and need not necessarily form a circumferential band.

FIG. 12 depicts a prosthetic liner 90 having different lengths of a solid inner surface to the liner or a solid thickness, in that such solid section is printed along side filaments or other structural elements permitting ventilation. The solid sections 95, 96, 97 are not perforated compared to sections outside of such solid sections. The different lengths or sections may be provided in different liners in that the solid section longitudinal lengths vary from liner to liner depending on limb length.

Regarding the solid patch length, in one embodiment, it is located to correspond to a user's groin area, particularly for a transfemoral amputee. Indeed, in a preferred embodiment, the solid patch intersects with the groin area to minimize skin irritation from rubbing (the solid patch does not rub perhaps as much as an apertured region of the liner may).

By providing a medical device according to embodiments described, the problems of medical devices such as liners poorly navigating the tension between mechanical strength needed to cushion and protect a body portion such as a residual limb and the need for a breathable device to mitigate the buildup of fluid and heat are addressed. The structures and methods for forming layers, multilayer filaments, and openings and structures defined advantageously provide for the permeability of the liner to fluid and heat while retaining needed structural strength to cushion the residual limb.

The embodiments of a liner further provide for a multilayer liner structure with layers and sub-layers that comprise different materials and/or properties for providing a liner with properly arranged portions having mechanical strength, elasticity, comfort features, frictional features, and stiffness.

It is to be understood that not necessarily all objects or advantages may be achieved under an embodiment of the disclosure. Those skilled in the art will recognize that the medical device may be embodied or carried out, so it achieves or optimizes one advantage or group of advantages as taught herein without achieving other objects or advantages as taught or suggested herein.

The skilled artisan will recognize the interchangeability of various disclosed features. Besides the variations described, other known equivalents for each feature can be mixed and matched by one of skill in this art to construct a medical device under principles of the present disclosure. It will be understood by the skilled artisan that the features described may apply to other types of orthopedic, prosthetic, or medical devices.

Although this disclosure describes certain exemplary embodiments and examples of a medical device or liner, it nevertheless will be understood by those skilled in the art that the present disclosure extends beyond the specifically disclosed prosthetic socket embodiments to other alternative embodiments and/or users of the disclosure and obvious modifications and equivalents thereof. It is intended that the present disclosure should not be limited by the particular disclosed embodiments described above, and may be extended to medical devices and supports and other uses that may employ the features described.

1. A system for additive manufacturing using uncured liquid silicone received from a reservoir, comprising:
a deposition apparatus comprising a nozzle, the deposition apparatus arranged to receive the uncured liquid silicone; and
a deposition substrate configured to receive the uncured liquid silicone deposited from the nozzle of the deposition apparatus, the deposition substrate having a cylindrical or conical mandrel defining an axis, the mandrel arranged to rotate about the axis thereof;
wherein the nozzle is adapted to move relative to the axis of the mandrel.

2. The system for additive manufacturing of claim 1, wherein the nozzle is arranged to be positioned offset relative to the axis.

3. The system for additive manufacturing of claim 1, wherein the deposition apparatus further comprises at least two nozzles.

4. The system for additive manufacturing of claim 3, wherein the at least two nozzles are arranged in a predetermined configuration and configured to deposit the uncured liquid silicone onto the mandrel simultaneously.

5. The system for additive manufacturing of claim 3, wherein the at least two nozzles move helically relative to the axis of the mandrel.

6. A prosthetic liner comprising:
an elastomeric body defined by a lattice structure including a first layer of first filaments discretely formed from a first elastomeric material and overlapping a second layer of second filaments discretely formed from a second elastomeric material, the first and second filaments of the first and second layers, respectively, overlapping and securing to one another at discrete intersections to form a first set of interstices located therebetween in a predetermined pattern;
wherein the first and second layers are blended at least in part with one another in a blended region at an interface of the first and second layers, the blended region forming a permanent chemical bond in which the first and second elastomeric materials of the first and second layers of the first and second filaments intermix;
an adhesive layer deposited onto the elastomeric body and layered onto at least a portion of an outermost surface of the elastomeric body.

7. The prosthetic liner of claim 6, wherein the adhesive layer is disposed in a pattern over the outermost surface differently from the elastomeric body.

8. The prosthetic liner of claim 7, wherein the adhesive layer has a discontinuous pattern disposed over the outermost surface of the elastomeric body.

9. The prosthetic liner of claim 6, further comprising a textile layer disposed over the adhesive layer, and secured to the elastomeric body by the adhesive layer.

10. The prosthetic liner of claim 9, wherein the textile layer has an end cap formed from an elastomeric material integrated therewith.

11. The prosthetic liner of claim 6, wherein the liner forms a proximal end and a distal end, and a body defined between the proximal end and distal end, and having a tubular shape defined about an axis of the body.

12. The prosthetic liner of claim 11, wherein the tubular shape is conical.

13. The prosthetic liner of claim 12, wherein the adhesive layer is disposed in a pattern over the outermost surface differently from the elastomeric body and in a discontinuous pattern relative to the axis of the body.

14. The prosthetic liner of claim 13, further comprising a textile layer disposed over the adhesive layer, and secured to the elastomeric body by the adhesive layer.

15. The prosthetic liner of claim 14, wherein the textile layer has an end cap formed from an elastomeric material integrated therewith.

16. The prosthetic liner of claim 15, wherein the elastomeric body defines at least a first recess at the distal end adapted to receive the end cap such that the end cap does not protrude beyond contours of the elastomeric body when secured thereon.

17. The prosthetic liner of claim 16, wherein the elastomeric body defines a second recess axially displaced from the first recess toward the proximal end.

18. The prosthetic liner of claim 17, wherein the second recess circumferentially extends about the axis of the elastomeric body.

19. The prosthetic liner of claim 18, further comprising a seal element having a first portion disposed in the second recess and a second portion extending outwardly relative to the axis and beyond the outermost surface of the elastomeric body.

20. A prosthetic liner comprising:
an elastomeric body defined by a lattice structure including a first layer of first filaments discretely formed from a first elastomeric material and overlapping a second layer of second filaments discretely formed from a second elastomeric material, the first and second filaments of the first and second layers, respectively, overlapping and securing to one another at discrete intersections to form a first set of interstices located therebetween in a predetermined pattern;
wherein the first and second layers are blended at least in part with one another in a blended region at an interface of the first and second layers, the blended region forming a permanent chemical bond in which the first and second elastomeric materials of the first and second layers of the first and second filaments intermix;
wherein the liner forms a proximal end and a distal end, and a body defined between the proximal end and distal end, and having a tubular shape defined about an axis of the body;
an adhesive layer deposited on an outermost surface of the elastomeric body and layered onto at least a portion of an outermost surface of the elastomeric body in a discontinuous pattern;
a textile layer disposed over the adhesive layer, and secured to the elastomeric body by the adhesive layer, the textile layer having an end cap formed from an elastomeric material integrated therewith;
wherein the elastomeric body defines at least a first recess at the distal end adapted to receive the end cap such that the end cap does not protrude beyond contours of the elastomeric body when secured thereon.
